(19) United States
(12) Patent Application Publication
(10) Pub. No.: US 2008/0032292 A1
(43) Pub. Date: Feb. 7, 2008

(54) Methods for the Detection of Ovarian Cancer

(76) Inventor: John Paul Cairns, Philadelphia, PA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 11/575,249

(22) PCT Filed: Sep. 14, 2005
(86) PCT No.: PCT/US05/32600

§ 371 Date: Aug. 1, 2007

Related U.S. Application Data
(60) Provisional application No. 60/610,041, filed on Sep. 15, 2004.

(51) Int. Cl. C12Q 1/68

(52) U.S. Cl. 435/6

(57) ABSTRACT

Methods and kits for the detection of ovarian cancer are provided.

This application claims priority under 35 U.S.C. §119(e) to U.S. Provisional Patent Application No. 60/610,041, filed on Sep. 15, 2004. The foregoing application is incorporated by reference herein.

Pursuant to 35 U.S.C. §202(c), it is acknowledged that the U.S. Government has certain rights in the invention described herein, which was made in part with funds from the National Institutes of Health, Grant Number P50 CA083638.

### FIELD OF THE INVENTION

This invention relates to the fields of oncology and molecular biology. More specifically, the present invention provides methods for detecting the presence of ovarian cancer based on the promoter methylation pattern of a pre-selected panel of genes.

### BACKGROUND OF THE INVENTION

Several publications and patent documents are cited throughout the specification in order to describe the state of the art to which this invention pertains. Each of these citations is incorporated herein by reference as though set forth in full.

There will be an estimated 25,580 new cases and 16,090 deaths from ovarian cancer in the US this year (1). The highly lethal nature of ovarian cancer is related to the absence of symptoms in the majority of women with early stages of the disease. Seventy per cent of patients have advanced disease (stage III or IV) upon presentation with a 5-year survival at best of 15-20% despite aggressive treatment. Yet when the cancer is detected early, women with stage I disease have a 5-year survival of 77-87% and with stage I tumors that are well-differentiated the 5-year survival is 94% (2). Current techniques to screen for ovarian cancer e.g. physical exam, CT scan, ultrasound, and the CA-125 serum marker have shown limited success (3-5). The BRCA1 and 2, site-specific ovarian cancer and hereditary nonpolyposis colorectal cancer (HNPCC) autosomal dominant familial syndromes account for an estimated 10% of ovarian cancer (6) and represent a high risk group for screening. Thus, new approaches to early detection of ovarian cancer are urgently needed since existing surgical and management methods can consistently cure only early stage cancer.

There is broad agreement that the genetic and epigenetic alterations which initiate and drive cancer can be potentially useful in the diagnosis and management of cancer (7). Silencing of tumor suppressor genes, such as p16^{INK4a}, VHL and the mismatch repair gene hMLH1, have established promoter hypermethylation as a common mechanism for tumor suppressor inactivation in human cancer and a promising new target for molecular detection (8, 9). Several cancer genes of clear biological significance, including p16^{INK4a} and BRCA1, have been found to have hypermethylation of normally unmethylated CpG islands within the promoter region in ovarian cancer cells (10-12). Hypermethylation can be analyzed by the sensitive methylation specific PCR (MSP) technique which can identify 1 methylated allele in 1000 unmethylated alleles (13), appropriate for the detection of few neoplastic cells in a background of normal cells. MSP also allows rapid analysis of multiple gene loci, does not require prior knowledge of epigenetic alteration and can potentially provide a “yes or no” answer for the detection of cancer (13, 14).

Bodily fluids that surround or drain the organ of interest from patients with various solid malignancies have been successfully used for MSP-based detection. These include detection of lung cancer in serum (15), sputum (16) and bronchial lavage (17), head and neck cancer in serum (18), breast cancer in ductal lavage (19) and prostate (20) or renal cancer (21) in urine. However, ovarian cancer has not yet been tested.

### SUMMARY OF THE INVENTION

In accordance with the present invention, a method for the detection of ovarian cancer is provided. An exemplary method entails providing a biological sample obtained from a patient and performing MSP on the nucleic acid molecules of the biological sample. The hypermethylation of the nucleic acids molecules obtained from the patient, in comparison to that from a normal subject, is indicative of ovarian cancer. According to one embodiment of the instant invention, the nucleic acid molecules comprise one or more tumor suppressor gene promoter regions. In a particular embodiment, at least one nucleic acid molecule of the biological sample is isolated prior to MSP. In yet another embodiment, a biological sample obtained from a normal subject can be analyzed along side the biological sample obtained from a patient. In still another embodiment, the MSP is performed on at least one tumor suppressor gene promoter region. In a particular embodiment of the instant invention, the at least one tumor suppressor gene promoter region comprises at least one tumor suppressor gene promoter region selected from the group consisting of the BRCA1, RASSF1A, APC, p14^{ARF}, p16^{INK4a} and DAP-kinase promoter regions. In another embodiment, the at least one tumor suppressor gene promoter region comprises at least the BRCA1 and RASSF1A promoter regions. In a particular embodiment, the at least one tumor suppressor gene promoter region comprises the BRCA1, RASSF1A, APC, p14^{ARF}, p16^{INK4a} and DAP-kinase promoter regions.

According to another aspect of the invention, kits for performing the methods described above are provided. Exemplary kits of the instant invention comprise at least one set of primers specific for performing methylation specific PCR of the promoter region of at least one of the genes selected from the group consisting of BRCA1, RASSF1A, APC, p14^{ARF}, p16^{INK4a} and DAP-kinase; and at least one hypermethylated nucleic acid molecule for use as a positive control or at least one agent (e.g., Sss I methylase) to methylate a nucleic acid molecule as a positive control. The kits may further comprise at least one unmethylated nucleic acid molecule for use as a negative control. The kits may also comprise nucleic acid molecules isolated from a normal subject wherein the nucleic acid molecules comprise the promoter region of at least one of the genes selected from the group consisting of BRCA1, RASSF1A, APC, p14^{ARF}, p16^{INK4a} and DAP-kinase. The kits of the instant invention may also comprise at least one of the following: reagents suitable for performing non-denaturing gel electrophoresis, reagents for performing MSP (for example, without limitation, sodium bisulfate, polymerase, dNTPs, buffers, and tubes), and instruction material.

### BRIEF DESCRIPTION OF THE DRAWINGS

FIGS. 1A and 1B are images of gels showing the MSP of BRCA1 and RASSF1A genes in ovarian tumor, peritoneal fluid (per fl), and serum DNAs (FIG. 1A) and in normal and benign disease control DNAs (FIG. 1B). Viewed from left to right, two patients and controls are shown in each gel panel in FIG. 1A. A tumor DNA with methylated alleles of BRCA1 or the tumor cell line MDA231 (RASSF1A) DNA was used as a positive control. Normal lymphocyte DNA was used as a negative control and a water control was used for contamination in the PCR reaction. A 20 bp molecular ruler (far left) is also provided as a molecular weight marker. M is methylated; U is unmethylated; NS is normal serum; NT is normal ovarian tissue; and BF is benign cystic disease.

### DETAILED DESCRIPTION OF THE INVENTION

Because existing surgical and management methods can consistently cure only early stage ovarian cancer, novel strategies for early detection are required. Silencing of tumor suppressor genes, such as p16^{INK4a}, VHL, and hMLH1, have established promoter hypermethylation as a common mechanism for tumor suppressor inactivation in human cancer and as a promising target for molecular detection in bodily fluids. Using sensitive methylation specific PCR, matched tumor, pre-operative serum or plasma, and peritoneal fluid (washes or ascites) DNAs obtained from 50 patients with ovarian or primary peritoneal tumors were screened for hypermethylation status of the normally unmethylated BRCA1 and RAS association domain family protein 1A (RASSF1A) tumor suppressor genes. Hypermethylation of one or both genes was found in 34 tumor DNAs (68%). Further examination of one or more of the adenomatous polyposis coli (APC), p14^{ARF}, p16^{INK4a} or death associated protein-kinase (DAP-Kinase) tumor suppressor genes revealed hypermethylation in each of the remaining 16 tumor DNAs which extended diagnostic coverage to 100%. Hypermethylation was observed in all histological cell types, grades, and stages of ovarian tumor examined. An identical pattern of gene hypermethylation was found in the matched serum DNA from 41 of 50 patients (82% sensitivity), including 13 of 17 cases of stage I disease. Hypermethylation was detected in 28 of 30 peritoneal fluid DNAs from stage IC-IV patients, including 3 cases with negative or atypical cytology. In contrast, no hypermethylation was observed in non-neoplastic tissue, peritoneal fluid, or serum from 40 control women (100% specificity). Thus, promoter hypermethylation is a common and relatively early event in ovarian tumorigenesis that can be detected in the serum DNA from patients with ovary-confined (stage IA or B) tumors and in cytologically negative peritoneal fluid. Analysis of tumor specific hypermethylation in serum DNA may enhance early detection of ovarian cancer.

The following definitions are provided to facilitate an understanding of the present invention. “Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, may refer to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism. Alternatively, this term may refer to a DNA that has been sufficiently separated from (e.g., substantially free of) other cellular components with which it would naturally be associated. “Isolated” is not meant to exclude artificial or synthetic mixtures with other compounds or materials, or the presence of impurities that do not interfere with the fundamental activity, and that may be present, for example, due to incomplete purification.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA molecule of the invention, to the substantial exclusion of hybridization of the oligonucleotide with single-stranded nucleic acids of non-complementary sequence. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (Sambrook et al., 1989):
Tₘ=81.5C+16.6 Log [Na+]+0.41(% G+C)−0.63 (% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5× Denhardt's solution, 0.5% SDS and 100 μg/ml denatured salmon sperm DNA at 42° C., and washed in 2×SSC and 0.5% SDS at 55° C. for 15 minutes. A high stringency hybridization is defined as hybridization in 6×SSC, 5× Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5× Denhardt's solution, 0.5% SDS and 100 μg/ml denatured salmon sperm DNA at 42° C., and washed in 0.1×SSC and 0.5% SDS at 65° C. for 15 minutes.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as appropriate temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

The term “gene” refers to a nucleic acid comprising an open reading frame encoding a polypeptide, including both exon and (optionally) intron sequences. The nucleic acid may also optionally include non coding sequences such as promoter or enhancer sequences. The term “intron” refers to a DNA sequence present in a given gene that is not translated into protein and is generally found between exons.

The term “promoter” or “promoter region” generally refers to the transcriptional regulatory regions of a gene. The “promoter region” may be found at the 5′ or 3′ side of the coding region, or within the coding region, or within introns. Typically, the “promoter region” is a nucleic acid sequence which is usually found upstream (5′) to a coding sequence and which directs transcription of the nucleic acid sequence into mRNA. The “promoter region” typically provides a recognition site for RNA polymerase and the other factors necessary for proper initiation of transcription.

The phrase “methylation specific polymerase chain reaction” refers to a simple rapid and inexpensive method to determine the methylation status of CpG islands. This approach allows the determination of methylation patterns from very small samples of DNA, including those obtained from paraffin-embedded samples, and can be used in the study of abnormally methylated CpG islands in neoplasia. Methylation-specific PCR is described, for example, in U.S. Pat. Nos. 5,786,146; 6,200,756; 6,017,704; and 6,265,171 and U.S. Patent Application Publication No. 2004/0038245.

The phrase “tumor suppressor genes” refers to a class of genes involved in different aspects of normal control of cellular growth and division, the inactivation of which is often associated with oncogenesis. The phrase “tumor suppressor genes” may also refer to those genes whose expression within a tumor cell suppresses the ability of such cells to grow spontaneously and form an abnormal mass, i.e., the expression of which is capable of suppressing the neoplastic phenotype and/or inducing apoptosis.

As used herein, the term “biological sample” refers to a subset of the tissues (e.g., ovarian tissue) of a biological organism, its cells, or component parts (e.g. body fluids such as, without limitation, blood, serum, plasma, and peritoneal fluid). In a preferred embodiment, the biological sample is selected from the group consisting of serum, plasma, and peritoneal fluid.

As used herein, an “instructional material” includes a publication, a recording, a diagram, or any other medium of expression which can be used to communicate the usefulness of the composition of the invention for performing a method of the invention. The instructional material of the kit of the invention can, for example, be affixed to a container which contains a kit of the invention to be shipped together with a container which contains the kit. Alternatively, the instructional material can be shipped separately from the container with the intention that the instructional material and kit be used cooperatively by the recipient.

The Example set forth below is provided to better illustrate certain embodiments of the invention. It is not intended to limit the invention in any way.

### EXAMPLE I

It was hypothesized that serum or plasma from patients with ovarian cancer might also contain hypermethylated DNA from tumor cells amenable to MSP analysis. In the instant study, a series of matched ovarian tumor, serum, and peritoneal fluid DNAs as well as normal and benign disease control DNAs was screened for aberrant promoter hypermethylation of BRCA1, RASSF1A and other tumor suppressor genes using the materials and methods set forth below.

Specimen Collection and Preparation

After approval from the Institutional Review Board, tumor or cyst tissue specimens was obtained via the Fox Chase Cancer Center (FCCC) Tumor Bank Facility and matched pre-operative serum or plasma via the FCCC Biospecimen Repository from 60 patients, aged 18 to 87 years, diagnosed with an ovarian or primary peritoneal lesion who underwent laparotomy or laparoscopy. Thirty-five patients had histologically verified ovarian tumors comprising 21 papillary serous, 3 mucinous, 4 clear cell, 5 endometroid, 1 transitional cell and 1 undifferentiated. Ten patients had borderline neoplasms of low malignant potential (LMP); 5 papillary serous, 4 mucinous and 1 mixed. Five patients had papillary serous tumors of primary peritoneal origin. Tumors were graded and staged according to American Joint Committee on Cancer guidelines (22). An additional 10 patients had benign ovarian cysts. Approximately 20-50 mls of ascites or peritoneal washing was aliquoted from routine collection for cytological analysis in 42 of the 50 cases. A further 21 archival stage I tumor specimens without matched fluid or serum were also obtained. Twenty serum specimens from normal healthy age-matched women were obtained via the FCCC Biospecimen Repository. Ten specimens of histologically normal (non-neoplastic) ovarian tissue were collected from the unaffected ovary in 2 cases of unilateral disease and from 8 female bladder cancer patients who underwent cystectomy.

Tumor tissue was obtained immediately after surgical resection and subsequently microdissected with the assistance of a pathologist. DNA was extracted from tissue, approximately 50 ml of peritoneal fluid, or 1.5 ml of serum using a standard technique of digestion with proteinase K in the presence of sodium dodecyl sulfate at 37° C. overnight followed by phenol/chloroform extraction (23). Tumor specimen DNA was spooled out after precipitation with 100% ethanol. Serum or peritoneal fluid DNA was precipitated with one-tenth volume of 10M ammonium acetate, 2 μl of glycogen (Roche Diagnostics Corporation, Indianapolis, Ind.) and 2.5 volumes of 100% ethanol, followed by incubation at −20° C. and centrifugation at top speed (16,000 RCF). Approximately 50 ng DNA was obtained from 1 ml of serum. For paraffin-embedded tissue, ten 7 μm sections were cut with a microtome and put on glass slides. A tumor cell-rich area or cyst, indicated by the pathologist, was removed with a razor blade, or needle depending on size, using an inverted microscope. The dissected tissue was placed directly into a microcentrifuge tube, washed with xylene, and DNA isolated as above.

Methylation Specific PCR

Specimen DNA (0.05-1 μg) was modified with sodium bisulfite, converting all unmethylated, but not methylated, cytosines to uracil followed by amplification with primers specific for methylated versus unmethylated DNA. The genes used for ovarian tumor cell DNA detection were BRCA1 (11), RASSF1A (24), APC (25), p14^{ARF} (26), p16^{INK4a} (13) and DAP-Kinase (27). The primer sequences used are set forth below.

(SEQ ID NO: 1)

| | |
|---|---|
| RASSF1A UF | GGG GTT TGT TTT GTG GTT TTG TTT |

(SEQ ID NO: 2)

| | |
|---|---|
| RASSF1A UR | AAC ATA ACC CAA TTA AAC CCA TAC TTC |

(SEQ ID NO: 3)

| | |
|---|---|
| RASSF1A MF | GGG TTC GTT TTG TGG TTT CGT TC |

(SEQ ID NO: 4)

| | |
|---|---|
| RASSF1A MR | TAA CCC GAT TAA ACC CGT ACT TCG |

(SEQ ID NO: 5)

| | |
|---|---|
| BRCA1 UF | TTG GTT TTT GTG GTA ATG GAA AAG TGT |

(SEQ ID NO: 6)

| | |
|---|---|
| BRCA1 UR | CAA AAA ATC TCA ACA AAC TCA CAC CA |

(SEQ ID NO: 7)

| | |
|---|---|
| BRCA1 MF | TCG TGG TAA CGG AAA AGC GC |

(SEQ ID NO: 8)

| | |
|---|---|
| BRCA1 MR | AAA TCT CAA CGA ACT CAC GCC G |

(SEQ ID NO: 9)

| | |
|---|---|
| APC UF | GTG TTT TAT TGT GGA GTG TGG GTT |

(SEQ ID NO: 10)

| | |
|---|---|
| APC UR | CCA ATC AAC AAA CTC CCA ACA A |

(SEQ ID NO: 11)

| | |
|---|---|
| APC MF | TAT TGC GGA GTG CGG GTC |

(SEQ ID NO: 12)

| | |
|---|---|
| APC MR | TCG ACG AAC TCC CGA CGA |

(SEQ ID NO: 13)

| | |
|---|---|
| p16 UF | TTA TTA GAG GGT GGG GTG GAT TGT |

(SEQ ID NO: 14)

| | |
|---|---|
| p16 UR | CAA CCC CAA ACC ACA ACC ATA A |

(SEQ ID NO: 15)

| | |
|---|---|
| p16 MF | TTA TTA GAG GGT GGG GCG GAT CGC |

(SEQ ID NO: 16)

| | |
|---|---|
| p16 MR | GAC CCC GAA CCG CGA CCG TAA |

(SEQ ID NO: 17)

| | |
|---|---|
| p14 UF | TTT TTG GTG TTA AAG GGT GGT GTA GT |

(SEQ ID NO: 18)

| | |
|---|---|
| p14 UR | CAC AAA AAC CCT CAC TCA CAA CAA |

(SEQ ID NO: 19)

| | |
|---|---|
| p14 MF | GTG TTA AAG GGC GGC GTA GC |

(SEQ ID NO: 20)

| | |
|---|---|
| p14 MR | AAA ACC CTC ACT CGC GAC GA |

(SEQ ID NO: 21)

| | |
|---|---|
| DAPK UF | GGA GGA TAG TTG GAT TGA GTT AAT GTT |

(SEQ ID NO: 22)

| | |
|---|---|
| DAPK UR | CAA ATC CCT CCC AAA CAC CAA |

(SEQ ID NO: 23)

| | |
|---|---|
| DAPK MF | GGA TAG TCG GAT CGA GTT AAC GTC |

(SEQ ID NO: 24)

| | |
|---|---|
| DAPK MR | CCC TCC CAA ACG CCG A |

The primers for RASSF1A include CpG site positions 7-9 on the forward primer and 13-15 on the reverse primer as described (24). PCR amplification of tumor DNA was performed for 31-37 cycles at 95° C. denaturing, 58-66° C. annealing and 72° C. extension with a final extension step of 5 minutes. Cycle number and annealing temperature depended upon the primer set to be used, each of which had been previously optimized for the PCR technology. In each set of DNAs modified and PCR amplified, a cell line or tumor with known hypermethylation as a positive control, normal lymphocyte or normal ovarian tissue DNA as a negative control, and water with no DNA template as a control for contamination were included. If no tumor cell line with known hypermethylation of a particular gene was available, normal human lymphocyte DNA in vitro methylated with Sss I methylase according to the manufacturers instructions (New England Biolabs, Beverly, Mass.) was used as a positive control. After PCR, samples were run on a 6% non-denaturing acrylamide gel with appropriate size markers and the presence or absence of a PCR product analyzed.

Statistical Analysis

The sensitivity of MSP-based detection of hypermethylation in peritoneal fluid or serum was calculated as number of positive tests/number of cancer cases. The specificity was calculated as number of negative tests/number of cases without cancer and in a second, distinct approach as number of negative tests/number of cases without hypermethylation of a particular gene. The association of tumor stage with positive detection of hypermethylation in serum or peritoneal fluid was assessed using Fisher's exact test. Results were considered statistically significant if the two-sided P value was ≦0.05.

### Results

The hypermethylation status of the normally unmethylated BRCA1 and RASSF1A tumor suppressor genes was examined in 50 ovarian or primary peritoneal tumor and matched serum and peritoneal fluid DNAs by the sensitive MSP assay which can detect 0.1% cancer cell DNA from a heterogenous cell population (13). The frequency of promoter hypermethylation of BRCA1 was 12 of 50 (24%) and RASSF1A 25 of 50 (50%) tumors. Thirty-four of the 50 (68%) tumor DNAs showed hypermethylation of one or both genes (Table 1). To increase the diagnostic coverage (whether a hypermethylated gene was available as a target in each case), the 16 tumors with unmethylated alleles of BRCA1 and RASSF1A were screened for hypermethylation of the APC, p14^{ARF}, p16^{INK4a} and DAP-Kinase tumor suppressor genes. All 16 tumors were found to have hypermethylated alleles of one or more of these genes (Table 1). Potential diagnostic coverage was further assessed in an additional 21 archive stage I tumor DNAs without matched serum or fluid. Twenty of 21, and therefore overall 70 (37/38 stage I, 33/33 stage III-IV) of 71 (99%) tumor DNAs showed hypermethylation of at least one of the 6 genes in the panel. Hypermethylation was observed in all histological cell types (papillary serous, mucinous, endometroid and clear cell), in all pathologic grades and stages of ovarian cancer examined including well-differentiated stage IA or B tumors, and in borderline neoplasms of low malignant potential (LMP). Thus, promoter hypermethylation of the tumor suppressor genes in the panel can be a relatively early event in ovarian tumorigenesis. Hypermethylation was found in patients of all ages. See Table 1 below.

TABLE 1
Clinicopathological/hypermethylation detection data of 50 ovarian cancer patients.

| | | | | | | RASSF1A | BRCA1 | Other Gene | |
|---|---|---|---|---|---|---|---|---|---|
| No. | Age | Cell Type | Grade | Stage | Cytology | T/PF/S | T/PF/S | T/PF/S | CA-125 |
| 1 | 54 | ps LMP | B | IA | n | U/U/U | U/U/U | M/U/M p14 | NA |
| 2 | 64 | ps LMP | B | IA | n | M/U/U | U/U/U | | 4 |
| 3 | 58 | muc LMP | B | IA | n | M/U/U | U/U/U | | 16 |
| 4 | 44 | ps LMP | B | IA | ND | M/—/U | U/—/U | | NA |
| 5 | 29 | muc LMP | B | IA | ND | U/—/U | U/—/U | M/—/M p16 | 2 |
| 6 | 45 | mix LMP | B | IA | n | M/U/M | U/U/U | | NA |
| 7 | 67 | muc LMP | B | IA | n | U/U/U | M/U/M | | 54 |
| 8 | 36 | muc LMP | B | IA | n | U/U/U | U/U/U | M/U/U APC | 102 |
| 9 | 28 | ps LMP | B | IIIB | y | U/U/U | U/U/U | M/M/M p16 | NA |
| 10 | 42 | ps LMP | B | IB | n | M/U/M | U/U/U | | 216 |
| 11 | 56 | endo | I | IA | ND | M/—/M | M/—/M | | 302 |
| 12 | 71 | endo | II | IA | n | M/M/M | U/U/U | | 1696 |
| 13 | 18 | muc | II | IA | n | M/U/M | U/U/U | | 44 |
| 14 | 68 | endo | II | IA | n | U/U/U | M/U/M | | 5 |
| 15 | 67 | cc | III | IA | n | M/U/M | U/U/U | | 25 |
| 16 | 57 | cc | III | IA | n | M/U/M | U/U/U | | 77 |
| 17 | 38 | endo | II | IC | y | M/M/M | M/M/M | | 548 |
| 18 | 62 | undiff | III | IC | y | U/U/U | U/U/U | M/M/M APC | NA |
| 19 | 54 | ps | III | III | y | U/U/U | M/M/M | | 2000 |
| 20 | 69 | ps | III | IIIB | y | U/U/U | U/U/U | M/M/M APC | 96 |
| 21 | 78 | ps | III | IIIC | ND | M/—/U | M/—/U | | 4739 |
| 22 | 60 | ps | I | IIIC | n | M/M/M | U/U/U | | 129 |
| 23 | 64 | ps | III | IIIC | ND | U/—/U | M/—/U | | 2849 |
| 24 | 34 | muc | I | IIIC | ND | M/—/M | U/—/U | | NA |
| 25 | 53 | ps | III | IIIC | ND | M/—/M | U/—/U | | 277 |
| 26 | 81 | ps | III | IIIC | y | U/U/U | M/M/U | | 8796 |
| 27 | 75 | muc | III | IIIC | y | U/U/U | M/M/M | | NA |
| 28 | 57 | ps | III | IIIC | y | U/U/U | U/U/U | M/U/M DAPK | NA |
| 29 | 53 | ps | III | IIIC | y | U/U/U | U/U/U | M/M/M p16 | 693 |
| 30 | 67 | ps | III | IIIC | y | U/U/U | U/U/U | M/M/M DAPK | 207 |
| 31 | 56 | ps & cc | III | IIIC | y | U/U/U | U/U/U | M/M/M p14 | 500 |
| 32 | 67 | ps & endo | III | IIIC | y | M/M/M | U/U/U | | 1785 |
| 33 | 39 | ps | II | IIIC | atypical | U/U/U | M/M/M | | 272.5 |
| 34 | 71 | ps | III | IIIC | y | U/U/U | M/M/M | | 80.5 |
| 35 | 68 | cc | III | IIIC | y | M/M/M | U/U/U | | NA |
| 36 | 50 | ps | NA | IIIC | atypical | U/U/U | U/U/U | M/M/M p16 | NA |
| 37 | 50 | endo & cc | III | IIIC | n | U/U/U | U/U/U | M/U/M p14 | 36 |
| 38 | 41 | ps | II | IIIC | y | M/M/M | U/U/U | | 11 |
| 39 | 69 | ps | III | IIIC | y | U/U/U | U/U/U | M/M/U APC | 96 |
| 40 | 62 | ps | II | IV | y | U/U/U | M/M/M | | 473 |
| 41 | 76 | ps | III | IV | y | M/M/M | U/U/U | | 732.9 |
| 42 | 72 | tc | III | IV | y | U/U/U | U/U/U | M/M/M APC | NA |
| 43 | 71 | cc | III | IV | ND | M/—/M | U/—/U | | NA |
| 44 | 62 | ps | III | IV | y | M/M/M | U/U/U | | 498 |
| 45 | 49 | ps | II | IV | y | U/U/U | U/U/U | M/M/M p16 | 289 |
| 46 | 75 | ps pp | III | III | y | U/U/U | U/U/U | M/M/M DAPK | 153 |
| 47 | 69 | ps pp | III | IIIC | y | M/M/M | U/U/U | | NA |
| 48 | 85 | ps pp | III | IIIC | y | M/M/M | U/U/U | | 413 |
| 49 | 78 | ps pp | III | IIIC | y | M/M/M | U/U/U | | 617 |
| 50 | 87 | ps pp | III | IV | y | M/M/M | U/U/U | | 1206 |

Age (years);
Cell Type, ps = papillary serous, muc = mucinous, cc = clear cell, endo = endometroid, tc = transitional cell, undiff. = undifferentiated, pp = primary peritoneal origin,
LMP = low malignant potential;
Grade = American Joint Committee on Cancer;
Stage = American Joint Committee on Cancer stage grouping;
M = methylated,
U = unmethylated;
Other Gene, only the gene methylated in the tumor and used as a target for detection in the matched serum and PF is listed. A CA-125 value >35 is considered abnormal and 0-35 as the normal range. Patient 9's LMP tumor had non-invasive implants outside the ovary and was therefore considered a stage III lesion;
NA = Not available.

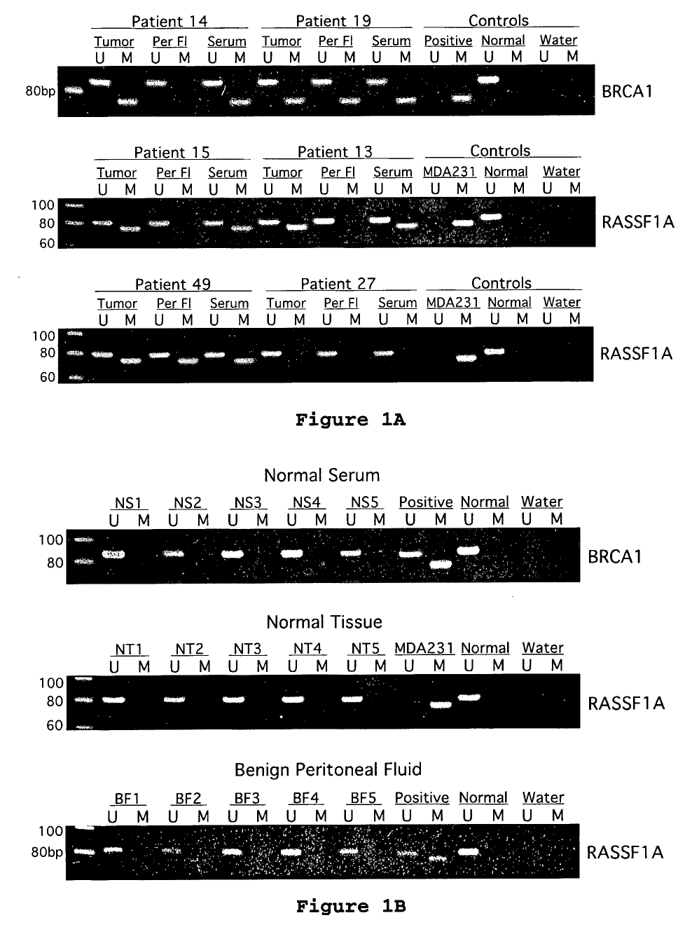

The MSP of BRCA1 and RASSF1A genes in ovarian tumor, peritoneal fluid, and serum DNAs are shown in FIG. 1A. Viewed from left to right, two patients are shown in each gel panel. In the BRCA1 gel panel, patient 14's stage IA tumor DNA is methylated. The methylation is absent in the peritoneal fluid DNA but positively detected in the serum DNA. Patient 19's tumor DNA is methylated with positive detection in both the peritoneal fluid and serum DNAs. In the top RASSF1A gel panel, both patient's 15 and 13 stage IA tumor DNAs are hypermethylated and positively detected in the corresponding serum DNAs, but absent in the peritoneal fluid DNAs. In the bottom RASSF1A gel panel, patient 49's tumor, peritoneal fluid, and serum DNA all show hypermethylation. Patient 27's tumor DNA is not methylated and the corresponding peritoneal fluid and serum DNA also show no hypermethylation. The PCR product in the unmethylated lane from all tumor DNAs arises from normal cell contamination of the tumor specimen or from an unmethylated allele. A tumor DNA with methylated alleles of BRCA1 or the tumor cell line MDA231 (RASSF1A) DNA were used as positive controls. Normal lymphocyte DNA was used as a negative control and a water control was used for contamination in the PCR reaction (right). The MSP of BRCA1 and RASSF1A genes in normal and benign disease control DNAs are shown in FIG. 1B. The absence of a PCR product in the methylated lane of BRCA1 in normal serum DNAs 1-5, RASSF1A in normal ovarian tissue DNAs 1-5, and in peritoneal fluid from patients with benign cystic disease DNAs 1-5 indicates that these specimen DNAs have unmethylated alleles only.

The hypermethylation status of the same genes in the matched serum and peritoneal fluid DNAs was determined and the pattern of gene hypermethylation found was compared to that of the corresponding tumor DNAs. An identical pattern of gene hypermethylation was detected in 41 of 50 (82%) matched serum or plasma DNAs (FIG. 1A and Table 1). The serum-positive cases included 13 of 17 cases of stage I and 28 of 33 stage III-IV tumors. No hypermethylation was detected in serum DNA from 9 (18%) patients. There was no statistical association between the tumor stage and positive detection in serum (13/17 stage I versus 28/33 stage III-IV, P=0.47, Fisher's exact test). Twenty-eight of the 30 stage IC-IV patient peritoneal fluid DNAs were MSP positive whereas 26 of 30 were cytologically positive (P=0.67, Fisher's exact test). One of the 12 peritoneal fluid DNAs from the stage IA or B patients showed methylation (patient 12, Table 1).

In contrast, hypermethylation of the gene panel was not observed in cyst tissue, serum or peritoneal fluid DNA from 10 patients with benign ovarian disease or in serum DNA from 20 normal, healthy age-matched women. Peritoneal fluid (PF) was available from only 8 of 10 patients with cystic disease. Hypermethylation was also absent in 10 normal (non-neoplastic) ovarian tissue DNAs (FIG. 1B and Table 2). Furthermore, a gene negative for hypermethylation in the tumor DNA was always negative in the matched serum or peritoneal fluid DNA, for example patient 27 in the RASSF1A gel panel shown in FIG. 1A. The specificity of the hypermethylated gene panel was therefore 100%.

TABLE 2
Hypermethylation detection data in 40 control women.

| | BRCA1 | RASSF1A | APC | p14 | P16 | DAP-K |
|---|---|---|---|---|---|---|
| Ovarian Cysts | 0/10 | 0/10 | 0/10 | 0/10 | 0/10 | 0/10 |
| Ovarian Cyst PF | 0/8 | 0/8 | 0/8 | 0/8 | 0/8 | 0/8 |
| Ovarian Cyst Serum | 0/10 | 0/10 | 0/10 | 0/10 | 0/10 | 0/10 |
| Normal Serum | 0/20 | 0/20 | 0/20 | 0/20 | 0/20 | 0/20 |
| Normal Ovary | 0/10 | 0/10 | 0/10 | 0/10 | 0/10 | 0/10 |

### Discussion

Successful detection of tumor specific aberrant hypermethylation in bodily fluids that surround or drain the organ of interest has been demonstrated in several tumor types (15-20), however ovarian cancer has yet to be tested. In many patients with ovarian cancer, tumor cells are present in peritoneal fluid by cytological examination. By definition, peritoneal fluid from stage IA and B cancer patients does not contain tumor cells by cytological examination (22), although it is not known whether free neoplastic DNA can be present. Molecular diagnosis in peritoneal fluid may be useful for early detection in high risk populations and also may complement traditional cytology for molecular staging. For the general population at risk of sporadic ovarian cancer, serum is a preferable choice of bodily fluid for molecular detection as it is readily accessible in all individuals from a peripheral blood sample, is currently used for CA-125 testing, and is enriched for tumor DNA in cancer patients (28). Several recent studies have shown that it is possible to detect tumor-specific genetic or epigenetic alterations in serum DNA from head and neck, lung and colon cancer patients (15, 18, 29). Importantly, tumor cell specific DNA alterations in serum were not limited to patients with metastatic cancer but were also present in serum from patients with early or organ-confined tumors (15, 18, 29). Neoplastic DNA in the serum most likely arises from cells that have left the site of the primary lesion and have invaded the circulatory system but lack the capacity of metastasis to new organs or may be released from the primary tumor as free DNA from nonviable (apoptotic) neoplastic cells (7).

The use of DNA-based methods for the early detection of ovarian cancer has several potential advantages. Since some genetic and epigenetic events will occur early in the disease process, molecular diagnosis may allow detection prior to symptomatic or overt radiographic manifestations. The epigenetic alteration of aberrant promoter hypermethylation can be detected at sensitive levels by PCR (1 in 1000) and importantly, since the alteration is a qualitative change, can provide a “yes or no” answer and is thus potentially very specific (13, 14).

Over 80% of ovarian cancer is of epithelial origin consisting of papillary serous, mucinous, endometroid, and clear cell histological cell types. There is also primary papillary serous carcinoma of the peritoneum, which is histologically identical to primary serous carcinoma of the ovary but is suspected to have a multifocal origin from the epithelial lining of the peritoneal cavity. A clinically distinct, intermediate form of epithelial ovarian cancer also exists, the ovarian tumor of low malignant potential (LMP) (6). The heterogeneity of gene alterations within and between distinct histological types mandates the use of a panel of genes. Indeed, no single gene is known to be hypermethylated in more than a proportion of ovarian tumors (10, 30, 31). It will likely be necessary to use a panel of genes to maximize detection of any type of adult sporadic cancer, analogous to the need for analysis of several genes for the diagnosis of familial breast cancer or HNPCC.

Until recently, the few genes identified as hypermethylated in ovarian cancer included GPC3 on the X chromosome (32), NOEY2 in an imprinted region of 1p (33) and myoD1 hypermethylated in ovarian tumors (34) but also reported to be methylated in normal tissue (35). Only genes hypermethylated in a cancer specific manner can be utilized in molecular detection strategies based on conventional MSP analysis. In addition to infrequent p16^{INK4a} hypermethylation (<10%), slightly more frequent hypermethylation (<15%) of two more genes, p14^{ARF} and DAP-Kinase, was found in ovarian tumors. Furthermore, hypermethylation of BRCA1 has been reported in 15-20% of sporadic ovarian tumors (11, 12) and a recent profile of hypermethylation reported RASSF1A to be hypermethylated in 41%, and APC in 18%, of ovarian cancer (31). Thus, it was timely to examine hypermethylation as a target for detection of ovarian cancer in bodily fluids.

Using the BRCA1, RASSF1A, APC, p14^{ARF}, p16^{INK4a} and DAP-Kinase tumor suppressor genes, it was demonstrated that promoter hypermethylation is common in ovarian cancer, including stage I disease, and can be readily detected in a specific manner in serum and peritoneal fluid DNAs. In this study, a sensitivity of 82% in serum was observed. Of interest was that methylation was detected in the serum DNA of 4 of 6 patients with CA-125 values of <35 (Table 1). Also, one non-neoplastic control patient with a fibroma had a CA-125 value of 63, but no methylation was detected in the paired serum DNA. Overall, hypermethylation was not detected in 9 (18%) serum DNAs from cancer patients. In these samples, neoplastic DNA may have been present in an amount lower than can currently be detected by conventional MSP. As is routine in PCR methodology, the PCR was limited to a maximum number of cycles (n=37) because it is known that specificity can decrease in MSP, as in other PCR protocols, with increased cycle number (36). It is possible that a higher number of cycles or a two-stage (nested) MSP approach (16) would have resulted in the positive detection of hypermethylation in the negative serum DNAs. No significant difference in detection frequency was observed between stage I disease and more advanced stage III-IV disease which suggested that tumor stage was not the main determinant of positive detection in serum. Hypermethylation was detected in 28, and cytology was positive in 26, of the 30 peritoneal fluids from stage IC-IV patients. Three peritoneal fluids with negative or atypical cytology were positive for hypermethylation (Patients 22, 33 and 36) however, one cytology positive fluid was negative for methylation (Patient 28). Hypermethylation in peritoneal fluid may be useful to accurately identify women that have a higher risk of developing recurrence and may be candidates for adjuvant therapy. Methylation was observed in only 1 peritoneal fluid from 15 stage IA or B patients but 11 of the 15 paired serums were positive for methylation. This suggests that free neoplastic DNA from ovary-confined disease accesses the bloodstream more readily than the peritoneum. The sensitivity of methylation-based detection may be improved by advances in collection techniques, enrichment of neoplastic cells or DNA from the fluid or serum by antibody or oligo-based magnetic bead technology, and improvements in PCR technology.

It is important that the target genetic alteration is cancer specific and not present in normal or benign cells. Although only genes reported to be unmethylated in normal cells were included in the hypermethylation panel, several controls to determine specificity were still performed. First, gene hypermethylation in cyst tissue, serum, and peritoneal fluid DNA from 10 patients with non-neoplastic ovarian disease or serum DNA from 20 normal, healthy controls was tested for, but none was observed (FIG. 1B). Second, the serum and peritoneal fluid DNAs were examined for the methylation status of a gene known to be unmethylated in the tumor DNA. This approach has been validated in previous MSP-based detection studies (15, 18, 20). There was no case where a serum or peritoneal fluid DNA gave a positive methylation result in the absence of methylation in the corresponding tumor (potential false positive) (Table 1). For example, tumor 27 in FIG. 1A did not have RASSF1A hypermethylation and the matched serum and peritoneal fluid DNAs were also negative. Third, 10 non-neoplastic ovarian tissue DNAs were examined and no hypermethylation was observed at the routine PCR amplification sensitivity (FIG. 1B). The findings in the 40 control women indicate that serum or peritoneal fluid hypermethylation is highly specific for cancer (Table 2). In addition, a recent report on the hypermethylation profile of ovarian cancer found no hypermethylation of BRCA1 or APC in 16 nonmalignant ovarian tissue specimens, although 2 specimens showed hypermethylation of RASSF1A (31). Primer sequences were used to different RASSF1A promoter CpG sites in the instant MSP analysis. A recent study reported DAP-Kinase hypermethylation in normal human lymphocytes by quantitative real-time MSP analysis (37). However, at the routine number of amplification cycles for conventional MSP, DAP-Kinase methylation was not observed in non-neoplastic DNAs (Table 2). The inclusion of several classical tumor suppressor genes, invariably inactivated in tumor cells only, as opposed to less well-defined cancer genes in the instant detection panel is likely one reason for the high specificity observed.

Genes that are hypermethylated exclusively, or more frequently, in ovarian cancer may be identified and included in an ovarian cancer detection panel to provide greater specificity for ovarian cancer (10, 38, 39). Algorithms may be used to score the specificity of a particular gene hypermethylation panel for the detection of ovarian cancer compared to other cancer types. At present, BRCA1 hypermethylation provides some specificity since this gene is methylated in breast and ovarian cancer only (11, 12). Furthermore, whether particular genes were methylated or not can aid in the prediction of the behavior of individual tumors within a particular pathologic stage. The heterogeneity of genetic alterations between tumors, for example which tumor suppressor gene pathways are abrogated in an individual tumor is likely one underlying cause of differences in tumor behavior and response to therapy. The panel employed here contained genes of clear biological significance such as the p16^{INK4a}, p14^{ARF} and APC genes involved in the p16/Rb and p53/p14 tumor suppressor gene pathways (40) and the Wnt signalling pathway (41), respectively. A recent report linking methylation of a Fanconi's anemia gene to cisplatinin sensitivity of ovarian cancer (42) indicates the potential of tumor profiling.

Molecular detection of loss of heterozygosity (LOH) or new alleles by microsatellite analysis has been reported in 17/20 (85%) serum and 12/19 (63%) peritoneal fluid DNAs from ovarian cancer patients (43), and by digital single nucleotide polymorphism (SNP) analysis in 19/20 (95%) ascitic fluids from ovarian carcinoma patients (44). Successful detection of p53 point mutation in matched peritoneal fluid from 3 patients has also been demonstrated (45), however p53 is not mutated in the majority of ovarian tumors (6). MSP-based detection has several advantages over microsatellite or point mutation-based detection of ovarian cancer. MSP has greater sensitivity which will be important for detection of early, small or precursor lesions. Also MSP, unlike point mutation, does not require prior knowledge of the gene status. At the protein level, telomerase-based detection was found to compare favorably with cytological examination of peritoneal fluid (46) and the potential of proteomic-based strategies for early detection has also been demonstrated (47). While the sensitivity of the MSP-based detection was lower than that reported in this proteomics study (47), the instant study detected alterations of identified tumor suppressor genes well characterized as biologically significant and known to be present in tumor cells. Different screening modalities and marker combinations, optimized for sensitivity and specificity, may be examined in concert for diagnosis of ovarian cancer.

The hypermethylation panel of 6 genes tested here provided almost 100% diagnostic coverage of 71 ovarian or primary peritoneal cancers, including all major histological cell types and pathologic stages, and is certainly manageable in terms of time and economy in view of current array and high-throughput technology. The potential of microarray technology for simultaneous screening for cancers of several different organ types may also partly address the issue that the relatively low incidence of ovarian cancer in the general population has been cited as one obstacle to screening for this disease (4, 5). In the near term, MSP-based detection could be used alongside an established marker, CA-125, to improve sensitivity and specificity. A typical 10 ml peripheral blood sample taken for CA-125 analysis would also provide enough serum for MSP analysis. In this study, it has been demonstrated for the first time the feasibility of hypermethylation-based, sensitive (82%) and 100% specific (no false positives) detection of ovarian cancer DNA in serum from patients with well-differentiated, organ-confined stage I tumors as well as advanced disease. Accordingly, hypermethylation has useful clinical application in ovarian cancer diagnosis and management.

### REFERENCES

1. Jemal A, Tiwari R C, Murray T, et al. Cancer Statistics, 2004. CA Cancer J Clin 2004;54: 8-29.
2. Vergote I, De Brabanter J, Fyles A, et al. Prognostic importance of degree of differentiation and cyst rupture in stage I invasive epithelial ovarian carcinoma. Lancet 2001;357:176-82.
3. Cannistra S A. Medical Progress: Cancer of the ovary. N. Engl. J. Med. 1993;329: 1550-59.
4. Runowicz CDaF, A. L. Screening for Gynecologic malignancies: A continuing responsibility. Surgical Oncology Clinics of North America 1999;8:703-24.
5. Bast R C, Jr. Status of tumor markers in ovarian cancer screening. J Clin Oncol 2003;21:200-5.
6. Dubeau L Ovarian Cancer, 2nd Ed. edition, Vol. Chapter 41, p. 675-80. New York: McGraw-Hill, 2002.
7. Sidransky D. Emerging molecular markers of cancer. Nat Rev Cancer 2002;2:210-9.
8. Baylin S B, Herman J G, Graff J R, Vertino P M Issa J-P J. Alterations in DNA methylation: a fundamental aspect of neoplasia. Adv. Cancer Res. 1998;72: 141-96.
9. Jones P A Laird P W. Cancer epigenetics comes of age. Nature Genet 1999;21: 163-67.
10. Esteller M, Corn P G, Baylin S B Herman J G. A gene hypermethylation profile of human cancer. Cancer Res. 2001;61:3225-29.
11. Esteller M, Silva J M, Dominguez G, et al. Promoter hypermethylation and BRCA1 inactivation in sporadic breast and ovarian tumors. J. Natl. Cancer Inst. 2000;92:564-69.
12. Baldwin R L, Nemeth E, Tran H, et al. BRCA1 promoter region hypermethylation in ovarian carcinoma: a population-based study. Cancer Res. 2000;60:5329-33.
13. Herman J G, Graff J R, Myöhänen S, B. D. N Baylin S B. Methylation-specific PCR: a novel PCR assay for methylation status of CpG islands. Proc Natl Acad Sci USA 1996;93:9821-26.
14. Baylin S B, Belinsky S A Herman J G. Aberrant methylation of gene promoters in cancer—concepts, misconcepts, and promise. J Natl Cancer Inst 2000;92: 1460-1.
15. Esteller M, Sanchez-Cespedes M, Rosell R, Sidransky D, Baylin S B Herman J G. Detection of aberrant promoter hypermethylation of tumor suppressor genes in serum DNA from non-small cell lung cancer patients. Cancer Res 1999;59:67-70.
16. Palmisano W A, Divine K K, Saccomanno G, et al. Predicting lung cancer by detecting aberrant promoter methylation in sputum. Cancer Res. 2000;60:5954-58.
17. Ahrendt S A, Chow J T, Xu L-H, et al. Molecular detection of tumor cells in bronchoalveolar lavage fluid from patients with early stage lung cancer. J Natl Cancer Inst 1999;91:332-39.
18. Sanchez-Cespedes M, Esteller M, Wu L, et al. Gene promoter hypermethylation in tumors and serum of head and neck cancer patients. Cancer Res 2000;60:892-95.
19. Evron E, Dooley W C, Umbricht C B, et al. Detection of breast cancer cells in ductal lavage fluid by methylation-specific PCR. The Lancet 2001;357:1335-36.
20. Cairns P, M. Esteller, J. G. Herman, M. Schoenberg, C. Jeronimo, M. Sanchez-Cespedes, N-H. Chow, M. Grasso, L. Wu, W. B. Westra and D. Sidransky. Molecular Detection of Prostate Cancer in Urine by GSTP1 Hypermethylation. Clinical Cancer Research 2001;7:2727-30.
21. Battagli C, Uzzo R G, Dulaimi E, et al. Promoter Hypermethylation of Tumor Suppressor Genes in Urine from Kidney Cancer Patients. Cancer Research 2003 ;63:8695-99.
22. Greene F L AJCC Cancer Staging Manual, 6th edition edition: Springer Verlag, 2002.
23. Sambrook J Russell D W Molecular Cloning. A Laboratory Manual. Cold Spring Harbor, N.Y.: Cold Spring Harbor Laboratory Press, 2001.
24. Dammann R, Li C, Yoon J-H, Chin P L, Bates S Pfeifer G P. Epigenetic inactivation of a RAS association domain family protein from the lung tumour suppressor locus 3p21.3. Nat. Genet. 2000;25:315-19.
25. Esteller M, Sparks A, Toyota M, et al. Analysis of adenomatous polyposis coli promoter hypermethylation in human cancer. Cancer Res 2000;60:4366-71.
26. Esteller M, Tortola S, Toyota M, et al. Hypermethylation-associated inactivation of p14(ARF) is independent of p16(INK4a) methylation and p53 mutational status. Cancer Res. 2000;60: 129-33.
27. Katzenellenbogen R A, Baylin S B Herman J G. Hypermethylation of the DAP-kinase CpG island is a common alteration in B-cell malignancies. Blood 1999;93:4347-53.
28. Chang H W, Lee S M, Goodman S N, et al. Assessment of plasma DNA levels, allelic imbalance, and CA 125 as diagnostic tests for cancer. J Natl Cancer Inst 2002;94: 1697-703.
29. Hibi K, Robinson C R, Booker S, et al. Molecular detection of genetic alterations in the serum of colorectal cancer patients. Cancer Res 1998;58: 1405-07.
30. Strathdee G, Appleton K, Illand M, et al. Primary ovarian carcinomas display multiple methylator phenotypes involving known tumor suppressor genes. Am J Pathol 2001;158:1121-7.
31. Rathi A, Virmani A K, Schorge J O, et al. Methylation profiles of sporadic ovarian tumors and nonmalignant ovaries from high-risk women. Clin Cancer Res 2002;8:3324-31.
32. Lin H, Huber R, Schlessinger D Morin P J. Frequent silencing of the GPC3 gene in ovarian cancer cell lines. Cancer Res. 1999;59:807-10.
33. Yu Y, Xu F, Peng H, et al. NOEY2 (ARHI), an imprinted putative tumor suppressor gene in ovarian and breast carcinomas. Proc. Natl. Acad. Sci. USA 1999;96:214-19.
34. Cheng P, Schmutte C, Cofer K F, Felix J C, Yu M C Dubeau L. Alterations in DNA methylation are early, but not initial, events in ovarian tumorigenesis. Br. J. Cancer 1997;75:396-402.
35. Rideout W M, 3rd, Eversole-Cire P, Spruck C H, 3rd, et al. Progressive increases in the methylation status and heterochromatinization of the myoD CpG island during oncogenic transformation. Mol Cell Biol 1994;14:6143-52.
36. Corn P G, Smith B D, Ruckdeschel E S, Douglas D, Baylin S B Herman J G. E-Cadherin expression is silenced by 5′ CpG island methylation in acute leukemia. Clin. Cancer Res. 2000;6:4243-48.
37. Reddy A N, Jiang W W, Kim M, et al. Death-associated protein kinase promoter hypermethylation in normal human lymphocytes. Cancer Res 2003;63:7694-8.
38. Costello J F, Frühwald M C, Smiraglia D J, et al. Aberrant CpG-island methylation has non-random and tumour-type-specific patterns. Nature Genet 2000;25: 132-38.
39. Wei S H, Chen C M, Strathdee G, et al. Methylation microarray analysis of late-stage ovarian carcinomas distinguishes progression-free survival in patients and identifies candidate epigenetic markers. Clin Cancer Res 2002;8:2246-52.
40. Sherr C J McCormick F. The RB and p53 pathways in cancer. Cancer Cell 2002;2: 103-12.
41. Taipale J Beachy P A. The Hedgehog and Wnt signalling pathways in cancer. Nature 2001;411:349-54.
42. Taniguchi T, Tischkowitz M, Ameziane N, et al. Disruption of the Fanconi anemia-BRCA pathway in cisplatin-sensitive ovarian tumors. Nat Med 2003;9:568-74.
43. Hickey K P, Boyle K P, Jepps H M, Andrew A C, Buxton E J Burns P A. Molecular detection of tumour DNA in serum and peritoneal fluid from ovarian cancer patients. Br. J. Cancer 1999;80:1803-08.
44. Chang H W, Ali S Z, Cho S K, Kurman R J Shih Ie M. Detection of allelic imbalance in ascitic supernatant by digital single nucleotide polymorphism analysis. Clin Cancer Res 2002;8:2580-5.
45. Parrella P, Zangen R, Sidransky D Nicol T. Molecular analysis of peritoneal fluid in ovarian cancer patients. Mod Pathol 2003;16:636-40.
46. Duggan B D, Wan M, Yu M C, et al. Detection of ovarian cancer cells: comparison of a telomerase assay and cytologic examination. J. Natl. Cancer Inst. 1998;90:238-42.
47. Petricoin E F, Ardekani A M, Hitt B A, et al. Use of proteomic patterns in serum to identify ovarian cancer. Lancet 2002;359:572-7.

While the invention has been described in detail and with reference to specific examples thereof, it will be apparent to one skilled in the art that various changes and modifications can be made therein without departing from the spirit and scope thereof.

What is claimed is:

1. A method for the detection of ovarian cancer, comprising,
a) providing a biological sample obtained from a patient;
b) performing methylation specific polymerase chain reaction on the nucleic acid molecules of said biological sample; and
c) comparing the methylation pattern of said nucleic acid molecules from said patient with the methylation pattern of said nucleic acid molecules from a normal subject,
wherein hypermethylation of said nucleic acid molecules obtained from said patient relative to the methylation pattern from said normal subject is indicative of the presence of ovarian cancer.

2. The method of claim 1, wherein said nucleic acid molecules comprise at least one promoter region of at least one tumor suppressor gene.

3. The method of claim 1, wherein said biological sample is selected from the group consisting of serum, plasma and peritoneal fluid.

4. The method of claim 1, wherein said nucleic acid molecules comprise at least one promoter region of at least one gene selected from the group consisting of BRCA1, RASSF1A, APC, p14^{ARF}, p16^{INK4a} and DAP-kinase.

5. The method of claim 1, wherein said nucleic acids comprise the promoter regions of the BRCA1, RASSF1A, APC, p14^{ARF}, p16^{INK4a} and DAP-kinase genes

6. The method of claim 1, wherein said patient has an ovary confined tumor.

7. The method of claim 1, further comprising isolating said nucleic acid molecules of said biological sample prior to performing the methylation specific polymerase chain reaction of step b).

8. The method of claim 1, wherein said methylation specific polymerase chain reaction comprises treating said nucleic acid molecules with sodium bisulfite prior to amplification.

9. The method of claim 1, further comprising performing methylation specific polymerase chain reaction on the nucleic acid molecules of a biological sample obtained from a normal subject.

10. A kit for practicing the method of claim 1, comprising
a) primers specific for performing methylation specific PCR of the promoter region of at least one of the genes selected from the group consisting of BRCA1, RASSF1A, APC, p14^{ARF}, p16^{INK4a} and DAP-kinase; and
b) at least one hypermethylated nucleic acid molecule for use as a positive control.

11. The kit of claim 10 further comprising at least one component selected from the group consisting of:
a) at least one unmethylated nucleic acid molecule for use as a negative control;
b) reagents suitable for performing methylation specific PCR;
c) reagents suitable for performing non-denaturing gel electrophoresis; and
d) instruction material.
